(19) United States
(12) Patent Application Publication
(10) Pub. No.: US 2010/0074944 A1
(43) Pub. Date: Mar. 25, 2010

(54) Transdermal Tobacco Alkaloid Reservoir Patch

(76) Inventors: Jesper Kruse Andersen, Valby (DK); Flemming Hjorth, Vejle (DK)

Correspondence Address: ST. ONGE STEWARD JOHNSTON & REENS, LLC, 986 BEDFORD STREET, STAMFORD, CT, 06905-5619, US

(21) Appl. No.: 12/627,986

(22) Filed: Nov. 30, 2009

Related U.S. Application Data
(63) Continuation of application No. PCT/DK2007/000257, filed on May 31, 2007.

(51) Int. Cl. A61K 9/70; A61K 31/465; A61P 25/34

(52) U.S. Cl. 424/449; 514/343

(57) ABSTRACT

The invention relates to a tobacco alkaloid patch (10; 41; 42; 43; 51; 52; 53; 54) for transdermal administration of a tobacco alkaloid, said patch comprising an impermeable backing (11) and a membrane (14) which defines a reservoir (12) there between, said membrane being permeable to and in contact with said tobacco alkaloid, said tobacco alkaloid being mixed with a viscous flowable gel, said impermeable backing and said membrane being at least partly joined by a sealing and wherein the amount of said tobacco alkaloid is above 10 mg.

### FIELD OF THE INVENTION

The invention relates to a transdermal reservoir patch according to claim 1.

### BACKGROUND OF THE INVENTION

The invention relates to a transdermal reservoir patch and in particular to a transdermal nicotine patch. The nicotine patches are generally intended to be applied for a regular and relatively constant maintenance of nicotine or a corresponding tobacco alkaloid in the blood of a user in order to avoid craving.

Transdermal devices for the delivery of a wide variety of physiologically active substances have been known for some time and transdermal devices in the form of reservoir patches are disclosed in e.g. U.S. Pat. No. 5,254,346 and in the form of matrix patches in e.g. U.S. Pat. No. 6,165,497. Such devices generally comprise an impermeable backing, a drug or physiologically active substance reservoir, a rate controlling membrane and an adhesive layer which by some means are sealed together to produce a transdermal delivery device.

Matrix designs, where drug provided as a semisolid with no membrane and drug-in-adhesive (DIA) are the dominant products on the market presently. The development of such technologies is relatively complex and costly. These designs require long-term compatibility among the drug, adhesive and excipients. Thus, the demands made on the adhesive may be somewhat stricter than those on reservoir systems. On the other hand, reservoir patches have certain disadvantages when applied as a tobacco alkaloid releasing patch compared to matrix or drug-in adhesive patches.

A general problem of matrix patches is that the area must be quite significant in order to deliver a desired amount of nicotine. Thus, some matrix patches has areas around 30 cm² in order to deliver 10 to 20 mg during a period of 16 hours.

Especially for matrix patches, the combination of the large dimensions of the patch and the stiffness might cause a higher risk of a corner of the patch releasing from the skin of the user and thereby causing nicotine to escape the skin of the user by evaporating into the air.

In order to overcome the problem of insufficient attachment of the patch the producers of matrix patches have added a larger amount of adhesive with a following extra irritation to the user.

A problem of e.g. applying a reservoir patch for nicotine delivery is that reservoir patches may have an even lower comfort level than matrix design, as the design would tend to be stiffer and less comfortable than corresponding matrix designs.

This problem is believed to be one of the reasons why nicotine reservoir patches have not found their way to market yet.

A further significant problem related to patches of the reservoir type is a higher risk of having a leakage from the patch; and in case of a leakage naturally more nicotine will be wasted in comparison to matrix and drug-in adhesive transdermal patches where the nicotine is bound e.g. in gels or other flux controlling substance.

In particular, this problem have proved significant when dealing with e.g. nicotine or nicotine derivates which are highly volatile and escape from the reservoir through even very small leakages even though such leakages are under very low pressure.

It is an object of the present invention to establish a patch suitable for release of a high dose of nicotine or corresponding tobacco alkaloid, which is secure and at the same time comfortable to the user.

### SUMMARY OF THE INVENTION

Reservoir patches are especially suitable for tobacco alkaloids and in particular for nicotine due to the possibility of delivering a high concentration of nicotine to the user.

The invention relates to a tobacco alkaloid patch (10; 41; 42; 43; 51; 52; 53; 54) for transdermal administration of a tobacco alkaloid, said patch comprising an impermeable backing (11) and a membrane (14) which defines a cavity (12) there between, said membrane being permeable to and in contact with said tobacco alkaloid, said impermeable backing and said membrane being at least partly joined by a sealing and wherein the amount of tobacco alkaloid is above 10 mg.

Said viscous flowable gel may work to immobilize said tobacco alkaloid between said impermeable backing and said membrane within said reservoir.

According to an embodiment of the invention, the amount of said tobacco alkaloid is above 20 mg.

According to an embodiment of the invention, the amount of said tobacco alkaloid is above 40 mg.

According to an embodiment of the invention, the amount of said tobacco alkaloid is above 60 mg, preferably above 80 mg, most preferably above 100 mg.

According to an embodiment of the invention, the initial flux of said tobacco alkaloid through the membrane is above 10 μg/cm² h.

According to an embodiment of the invention, the initial flux of said tobacco alkaloid through the membrane is above 50 μg/cm² h.

According to an embodiment of the invention, it is now possible to obtain patches with a tobacco alkaloid flux higher than the prior art patches. Two patches presently on the market both have fluxes of approximately 30 μg/cm² h. These two are a drug in adhesive and a matrix patch respectively and with the use of a reservoir patch according to embodiments of the present invention it is possible to reach a higher flux.

Throughout the description the initial flux should be understood as the flux immediately after releasing the liner and applying the patch on the body. For all practical purposes it is easier to calculate an average flux of the patch, i.e. a flux that may be calculated as the nicotine amount to be released divided by the delivering area and the amount of time during which the amount is to be released. However, due to the structure of the reservoir patch, the tobacco alkaloid release profile is usually smooth and the flux over an effective period, starting with the initial flux, will be reasonably constant and close to the average flux.

According to an embodiment of the invention, the initial flux of said tobacco alkaloid through the membrane is above 100 μg/cm² h.

According to an embodiment of the invention, the initial flux of said tobacco alkaloid through the membrane is above 1000 μg/cm² h.

According to an embodiment of the invention, the initial flux of said tobacco alkaloid through the membrane is above 2500 μg/cm² h.

According to an embodiment of the invention, the ratio between said tobacco alkaloid in the patch and the patch delivering area is above 1 mg/cm².

The term “tobacco alkaloid in the patch” covers the tobacco alkaloid present in the patch as a whole, i.e. all that is in the reservoir, the membrane and adhesive. For a tobacco alkaloid reservoir patch it is to be expected that at any time the most of the tobacco alkaloid is present in the reservoir.
The term “patch delivering area” is to be interpreted as the area of skin above which tobacco alkaloid is present, i.e. the total patch area minus the area of sealing.

According to an embodiment of the invention, the ratio between said tobacco alkaloid in the patch and the patch delivering area is above 2.5 mg/cm².

According to an embodiment of the invention, the ratio between the delivered tobacco alkaloid and the patch delivering area is above 0.5 mg/cm².

According to an embodiment of the invention, the ratio between the delivered tobacco alkaloid and the patch delivering area is above 1 mg/cm².

According to an embodiment of the invention, a higher delivered amount of tobacco alkaloid per delivering area is possible. Two patches presently on the market have delivered tobacco alkaloid per delivering area of 0.5 mg/cm² and 0.9 mg/cm² respectively. These two are a drug in adhesive and a matrix patch respectively and with the use of a reservoir patch according to embodiments of the present invention it is possible to have a higher amount of tobacco alkaloid delivered per delivering area.

According to an embodiment of the invention, the ratio between the delivered tobacco alkaloid and the patch delivering area is above 1.5 mg/cm².

According to an embodiment of the invention, the ratio between the delivered tobacco alkaloid and the patch delivering area is above 2.5 mg/cm².

According to an embodiment of the invention, the nicotine permeability of said membrane is above 20 μg·100 μm/cm²·hr.

According to an embodiment of the invention, the nicotine permeability of said membrane is above 50 μg·100 μm/cm²·hr.

According to an embodiment of the invention, the nicotine permeability of said membrane is above 220 μg·100 μm/cm²·hr.

According to embodiments of the invention, an improved low-area tobacco alkaloid patch has been obtained. The patch according to embodiments of the invention benefits from high dose, low leakage and high user comfort.

According to an embodiment of the invention, said sealing comprises a glue sealing.

According to an embodiment of the invention, said sealing comprises a heat sealing.

According to an embodiment of the invention, the width of said sealing is at least 1.5 mm.

It is advantageous to keep the width of the sealing above a certain value in order to avoid leakage of reservoir content through the adhesive or through holes in the patch caused by a separation of the layers due to a too low seal width.

According to an embodiment of the invention, the width of said sealing is at least 2.0 mm.

According to an embodiment of the invention, the width of said sealing is at most 10.0 mm.

It has been realized that an increased sealing width induces very few benefits, if any, as an increased sealing width causes the reservoir patch to be less comfortable to wear for the user due to the higher stiffness all around the reservoir patch. A higher stiffness like this will also increase the risk of a corner or a side of the patch releasing from the skin of the user when placed on a curving surface as for instance an arm or a leg.

Therefore, a sealing width will be problematic both when it is produced either too big or too small.

According to an embodiment of the invention, said membrane and said backing is joined at the circumference of the patch.

According to an embodiment of the invention, said sealing comprises circumferential sealing plus one or more sealing dots therein between.

A number of dot-formed sealings within the center of the patch will stabilize the patch and ensure a more equal distribution of gel and tobacco alkaloid.

According to an embodiment of the invention, said sealing comprises a circumferential sealing plus a pattern of any kind.

According to an embodiment of the invention, said patch comprises a detachable release liner (16).

In an embodiment of the invention, a detachable release liner is provided in order to protect the adhesive layer and retain said tobacco alkaloid prior to use.

According to an embodiment of the invention, said detachable release liner comprises one or more peelable corners.

According to an embodiment of the invention, the detachable release liner is attached to the membrane of the patch by an adhesive force which is weaker than the adhesive force obtained by said sealing between said membrane and said backing.

According to an embodiment of the invention, said tobacco alkaloid comprises nicotine.

The term “tobacco alkaloid” as used herein and in the claims, is taken to mean nicotine or nicotine-like alkaloid such as nor-nicotine, lobeline, and the like, in the free base or pharmacologically acceptable acid addition salt form. Plant alkaloids of this type are obtainable from species of Nicotiana, which is a source for nicotine and nor-nicotine, as well as species of Lobelia and Lobeliaceae (Indian tobacco) which are sources for lobeline.

According to an embodiment of the invention, said patch delivers more than about 50% of the total content of tobacco alkaloid to the user within 24 hours from releasing the liner and placing the patch on the skin of the user.

According to an embodiment of the invention, said patch delivers more than about 60% of the total content of tobacco alkaloid to the user within 24 hours from releasing the liner and placing the patch on the skin of the user.

According to an embodiment of the invention, said patch delivers more than about 70% of the total content of tobacco alkaloid to the user within 24 hours from releasing the liner and placing the patch on the skin of the user.

According to an embodiment of the invention, said impermeable backing further comprises permeable layers, said layers may be placed on the inner side and/or on the outer side of the impermeable backing.

According to an embodiment of the invention, said impermeable backing is made of a multilayer film.

According to an embodiment of the invention, said impermeable backing is made of a multilayer polyester film.

According to an embodiment of the invention, said impermeable backing is made of combinations of one or more of the following: pigmented polyolefin, aluminized polyester, metal foil and heat-sealable polyolefinic layers.

According to an embodiment of the invention, said impermeable backing has a thickness between 1 μm and 500 μm.

According to an embodiment of the invention, said impermeable backing has a thickness between 5 μm and 200 μm.

According to an embodiment of the invention, said impermeable backing has a thickness between 10 μm and 100 μm.

According to an embodiment of the invention, said membrane comprises a polyethylene membrane.

According to an embodiment of the invention, said membrane comprises polyamides, such as nylon 6,6, or some grades of ethylene vinyl acetate copolymers or functional equivalents of these.

According to an embodiment of the invention, said membrane has a thickness between 1 μm and 500 μm.

According to an embodiment of the invention, said membrane has a thickness between 5 μm and 200 μm.

According to an embodiment of the invention, said membrane has a thickness between 10 μm and 100 μm.

According to an embodiment of the invention, said membrane is non-porous.

According to an embodiment of the invention, said patch comprises an adhesive.

In an embodiment of the invention, said membrane of said patch is faced with an adhesive, e.g. a tape, which may be applied to mount the patch fixedly on the skin of a user.

According to an embodiment of the invention, said patch comprises an adhesive substantially equally distributed all over the lower part of the patch.

According to an embodiment of the invention, said patch comprises an adhesive pattern.

An adhesive pattern may be any possible pattern. Examples of adhesive distribution beneath the patch to be mentioned here are; a circumferential rim around the patch, a circumferential rim plus a central point, a circumferential rim and one or more crossing lines, a circumferential rim and one or more circular rims therein between.

According to an embodiment of the invention, said adhesive has a thickness of below 3 mm.

According to an embodiment of the invention, said adhesive has a thickness of below 1 mm.

According to an embodiment of the invention, said adhesive has a thickness of above 0.01 μm.

According to an embodiment of the invention, said adhesive has a thickness of above 0.1 μm.

According to an embodiment of the invention, an adhesive layer is attached to said membrane in order to attach the patch to the skin of a user.

According to an embodiment of the invention, said cavity forms a reservoir for said tobacco alkaloid.

According to an embodiment of the invention, said tobacco alkaloid is contained within said reservoir in liquid form.

According to an embodiment of the invention, said tobacco alkaloid is confined between said impermeable backing and said membrane within said reservoir substantially immobilized by a viscous flowable gel.

The gel comprises e.g. purified water and a gelling agent in a suitable distribution in order to obtain an appropriate viscosity.

In an embodiment of the invention, an added dose may be provided when the user activates the patch. In this way the user receives a normal “low” dose of nicotine throughout the day and can activate the patch to give an extra dose if craving symptoms arise. The activation can be performed by e.g. pressing the patch or in any manual or automatic way.

In an embodiment of the invention, a patch is provided comprising a combination of a matrix patch and a reservoir patch in order to utilize advantages from both.

According to an embodiment of the invention, said reservoir contains tobacco alkaloid in an amount of more than 0.5 mg.

According to an embodiment of the invention, said reservoir contains a gelling agent in an amount of less than 20 mg.

According to an embodiment of the invention, said reservoir contains a gelling agent in an amount of less than 10 mg.

According to an embodiment of the invention, said reservoir contains purified water in an amount of less than 1000 mg.

According to an embodiment of the invention, said reservoir contains purified water in an amount of less than 800 mg.

According to an embodiment of the invention, said reservoir contains tobacco alkaloid, gelling agent and purified water in a total amount of less than 1000 mg.

According to an embodiment of the invention, said reservoir contains tobacco alkaloid, gelling agent and purified water in a total amount of less than 800 mg.

According to an embodiment of the invention, said reservoir is shaped in a rectangular, circular or oval form.

According to an embodiment of the invention, said reservoir is made up of several minor reservoirs separated by sealing.

According to an embodiment of the invention, said reservoir is shaped in the form of a donut.

According to an embodiment of the invention, said reservoir is shaped in an essentially rectangular form with rounded corners.

According to an embodiment of the invention, said reservoir has a volume of less than 4000 mm³, preferably less than 3000 mm³.

According to an embodiment of the invention, said reservoir has a volume of less than 2000 mm³, preferably less than 1500 mm³.

According to an embodiment of the invention, said reservoir has a volume of more than 50 mm³, preferably more than 75 mm³.

According to an embodiment of the invention, said patch can be separated into two or more separate reservoir patches.

By tearing the patch along the peel line, two or more patches that can work individually are provided. The main patch alone or each patch may be provided with a peel corner.

According to an embodiment of the invention, the border region(s) between said two or more reservoir patches comprises predefined peel-lines.

According to an embodiment of the invention, the corners of said two or more separate reservoir patches are pre-rounded.

The patches may be pre-rounded by shaping of the reservoir patch(es) during manufacturing in such way that at least one of the patches are substantially free of sharp corners when peeled of and positioned on the skin of a user.

According to an embodiment of the invention, said patch can be separated into two or more separate patches with rounded corners.

According to an embodiment of the invention, said reservoir comprise a flux controlling gel.

According to an embodiment of the invention, said gel is constituted by purified water and a gelling agent selected from the group of hyroxyethyl starch, dextran, hydroxyethylcellulose (HEC), hydroxypropylmethylcellulose (HPMC), hydroxypropycellulose (HPC), methylcellulose (MC), hydroxyethyl-methylcellulose (HEMC), ethylhydroxyethylcellulose (EHEC), carboxymethyl cellulose (CMC), poly(vinyl alcohol), poly(ethylene oxide), poly(2-hydroxyethylmethacrylate), pyrolidone), and pluronics.

According to an embodiment of the invention, said gelling agent is hydroxypropyl methyl cellulose.

According to an embodiment of the invention, said gelling agent is methyl cellulose.

According to an embodiment of the invention, said patch is colored in order to match the skin of a user.

According to an embodiment of the invention, said patch is essentially transparent.

According to an embodiment of the invention, said patch is produced with a pattern or a picture on the backing.

According to an embodiment of the invention, the total size of said patch is less than 40 cm².

It is an advantage of the reservoir patch according to embodiments of the invention that it is possible to increase the amount of tobacco alkaloid without having to drastically increase the size of the patch with a following decreased flexibility. Hence, the reservoir patch is particularly suitable for high-dose patches, compared to matrix patches and drug-in-adhesive patches where a high-dose patch is equivalent to a large-sized patch.
Due to the better utilization of nicotine in the reservoir patches compared to drug-in-adhesive patches and matrix patches, it is possible with reservoir patches to make a high-dose patch capable of delivering a large amount of nicotine.

The reservoir patch according to an embodiment of the invention is capable of containing very high amounts of nicotine or tobacco alkaloid as compared to e.g. drug-in-adhesive patches, where high amounts of e.g. nicotine in the adhesive may destroy the adhesive or make a reaction therewith due to the fact that all nicotine must be effectively contained in the adhesive. Destruction of the adhesive naturally results in loosening of the patch and a reaction between the adhesive and the tobacco alkaloid may both loosen the patch and/or cause that the amount of tobacco alkaloid released is not as high as intended.

According to an embodiment of the invention, the total size of said patch is less than 25 cm².

According to an embodiment of the invention, the total size of said patch is above 1 cm².

According to an embodiment of the invention, the total size of said patch is above 2 cm².

According to an embodiment of the invention, said patch is rectangular, circular or oval.

Any patch shape suitable for defining a cavity between an inner and an outer surface is according to the invention.

According to an embodiment of the invention, the flexural strength of said patch is below 50 mN/mm.

According to an embodiment of the invention, said reservoir further contains another active ingredient.

According to an embodiment of the invention, one or more enhancers are added to enhance the uptake of said other active ingredient.

According to an embodiment of the invention, said enhancers are present in an amount of less than 2000 mg.

Different physiologically active substances show very varying ability to penetrate the skin. Nicotine easily permeates the skin, whereas other physiologically active substances, typically larger in molecule size, must be assisted in order to get a noticeable amount of ingredient through the skin. For this purpose enhancers are used in an embodiment of the invention.

Suitable penetration enhancers (flux enhancers are preferably monovalent, saturated or unsaturated aliphatic, cycloaliphatic or aromatic alcohols having from 4 to 12 carbon atoms, e.g. n-hexanol or cyclohexanol, aliphatic, cycloaliphatic or aromatic hydrocarbons having from 5 to 12 carbon atoms, e.g. hexane, cyclohexane, isopropylbenzene and the like, cyclo-aliphatic or aromatic aldehydes and ketones having from 4 to 10 carbon atoms, such as cyclohexanone, acetamide, N,N-di-lower alkylacetamides such as N,N-dimethylacetamide or N,N-diethyl-acetamide, c.sub.10-c.sub.20-alkanoylamides, e.g. N,N-dimethyllauroylamide, 1-n-C.sub.10-c.sub.20-alkylazcycloheptan-2-one, e.g. 1-n-dodeclyazacycloheptan-2-one(Azone.RTM. laurocapram), or N-2-hydroxyethylacetamide, and known vehicles and/or penetration enhancers such as aliphatic, cycloaliphatic and aromatic esters N,N-di-lower alkylsulphoxides, unsaturated oils, halogenated or nitrated aliphatic or cyclo-aliphatic hydrocarbons, salicylates, polyalkylene glycol silicates, and mixtures thereof.

The patches according to embodiments of the invention have elasticity suitable to follow the skin of the user. As the seal width will be limiting for the elasticity of the patch, the ability to lower the seal width, increases the elasticity and hence the comfort for the user.

In an embodiment of the invention, the Young Modulus of the patches is below 50 GPa irrespective of seal width.

According to an embodiment of the invention, said patch comprising an impermeable backing (17) and a membrane (14) which defines a reservoir (12) there between, said membrane (14) being permeable to and in contact with said tobacco alkaloid, said impermeable backing (17) and said membrane (14) being at least partly joined by a sealing (11), wherein said patch has a flexural strength of less than about 250 mN/mm.

According to an advantageous embodiment of the invention, high-dose nicotine patches may be obtained having acceptable membrane areas and featuring acceptable user comfort.

According to an advantageous embodiment of the invention, a high degree of flexibility, low flexural strength, is advantageous. This is in particular the case for people moving around a lot, e.g. craftsmen or during sports or generally relevant to people who desire freedom to move unrestricted.

In an embodiment of the invention, said patch has a flexural strength of less than 150 mN/mm.

In an embodiment of the invention, said patch has a flexural strength of less than 100 mN/mm.

In an embodiment of the invention, said patch has a flexural strength of less than 70 mN/mm.

In an embodiment of the invention, said patch comprising an impermeable backing (17) and a membrane (14) which defines a reservoir (12) there between, said membrane (14) being permeable to and in contact with said tobacco alkaloid, said impermeable backing (17) and said membrane (14) being at least partly joined by a sealing (11), wherein said patch has a flexural strength of less than about 50 mN/mm.

According to embodiments of the invention, it has been established that the flexural strength comparable and superior to matrix patches may be obtained through a proper design by basic design parameters, and it has moreover been established that bending rigidity may be relatively easily adjusted e.g. by the choice of sealing pattern without compromising the choice of materials.

Specifically, it has been obtained according to embodiments of the invention that secure and comfortable high-dose nicotine patches may be obtained by applying a reservoir type patch as delivery system.

Thus, it has been established that the critical backing layer may e.g. be increased in thickness or modified in structure in order to strengthen the resulting protection from the backing if at the same time the sealing pattern is designed to keep the overall flexural strength low.

A further advantage in keeping the flexural strength low is that the leakage of nicotine from the reservoir is minimized due to a reduced risk of breaking the patch during bending and/or a reduced risk of leakage due to partial release of the patch along the edge or corners when mounted on the skin of a user.

A further and important advantage of the invention is that high dosage nicotine may be obtained even when increasing the size of the patch as long as the flexural strength is kept below about 50 mN/mm, preferably below 40 mN/mm without compromising user comfort and especially the strict non-leakage requirements related to encapsulation of nicotine or corresponding alkaloids.

In particular, it has been established that leakage both from and into the patch via a membrane, i.e. leakages due to insufficient mounting on the skin of a user, has been minimized when minimizing the flexural strength of the complete patch.

According to a preferred embodiment of the invention, flexural strength refers to the ability of the patch to “bend” when mounted on the skin of a user.

In particular, the obtained tobacco alkaloid releasing patch has demonstrated high-dose application and high protection against intruding air or humidity in the interface between patch and the skin of a user. Moreover, a minimum of leakages in the backing or in the sealing has been obtained due to the flexible construction.

In an embodiment of the invention, said impermeable backing (17) has a flexural strength which is greater than the flexural strength of the membrane (14).

In an embodiment of the invention, said impermeable backing has a flexural strength of at least 0.5 mN/mm.

In an embodiment of the invention, said impermeable backing has a flexural strength of at least 1 mN/mm.

According to a preferred embodiment of the invention, the flexural strength must be at least 1 mN/mm in order to ensure proper protection of the membrane during use. Thus, the backing should be designed to a minimum of flexural strength in order to avoid leakage and damaging of the membrane.

In an embodiment of the invention, the flexural strength of the impermeable backing is at least 0.5 mN/mm and said patch has a flexural strength of less than about 50 mN/mm.

In an embodiment of the invention, the flexural strength of the impermeable backing is at least 0.5 mN/mm and said patch has a flexural strength of less than about 40 mN/mm.

In an embodiment of the invention, the impermeable backing of said patch has a flexural strength of at least less than about 30 mN/mm.

In an embodiment of the invention, said patch has a flexural strength of less than about 25 mN/mm.

In an embodiment of the invention, said patch has a flexural strength of about 1 to 30 mN/mm.

According to a preferred embodiment of the invention, the flexural strength of the nicotine releasing patch should be within a certain desired range in order to obtain an advantageous combination of patch strength, secure patch attachment and user comfort.

In an embodiment of the invention, the backing (17) comprises a multilayer structure.

In an embodiment of the invention, said tobacco alkaloid patch is a nicotine reservoir patch.

In an embodiment of the invention, said patch has a flexural strength greater than about 2 mN/mm.

According to a preferred embodiment of the invention, certain rigidity is required in order to facilitate a proper distribution of (an) active substance(s), specifically the tobacco alkaloid. When having certain rigidity, the reservoir may maintain a constant or at least reasonable stable form.

Moreover, handling requires a minimum of rigidity when positioning the patch on the user.

According to an advantageous embodiment of the invention, it has been shown that a sealing width too low will cause a risk of leakage of nicotine between the backing layer and the membrane through the sealing. When the width of the sealing is lowered, the sealing will decrease in quality. On the other hand it has also been realized that the width of a sealing may actually be produced lower than expected still featuring a secure sealing. This is partly believed to reside in the fact that a decreased sealing width invokes a higher flexibility in the overall path, which in turn results in a strengthened sealing.

In an embodiment of the invention, the amount of said tobacco alkaloid is above 110 mg, preferably above 160 mg, most preferably above 210 mg.

In an embodiment of the invention, said patch has a (reservoir volume)/(delivering area) ratio of more than 1 mm³/cm².

In an embodiment of the invention, said tobacco alkaloid patch is packed in a controlled atmosphere.

With the tobacco alkaloid patch packed in a controlled atmosphere improved properties may be obtained with regard to ensuring storage life of the patches. Preferably the patch is held in a controlled atmosphere from manufacturing till mounting on the user. Preferred gasses to be used as controlled atmosphere are inert gasses in order to prevent reactions with the tobacco alkaloid.
When e.g. oxygen is present in amounts similar to oxygen in the atmosphere the nicotine may degrade or react with oxygen to reduce the effect of the patch.

In an embodiment of the invention, said controlled atmosphere is nitrogen.

### THE DRAWINGS

The invention will now be described with reference to the drawings of which

FIG. 1 illustrates a top view of an embodiment of the invention,

FIG. 2a illustrates a side view of said embodiment of the invention, with

FIG. 2b illustrating a magnification of FIG. 2a in order to indicate the different elements,

FIG. 2c illustrating the patch of FIG. 2b after releasing the liner and being placed on a surface,

FIGS. 3a, 3b, 3c, 4a, 4b, 4c, 5a, 5b, 5c, 5d, 5e and 5f illustrate further embodiments according to embodiments of the invention, and

FIGS. 6a and 6b illustrate how a measurement of the flexural strength of a patch or an element of the patch is performed.

### DETAILED DESCRIPTION

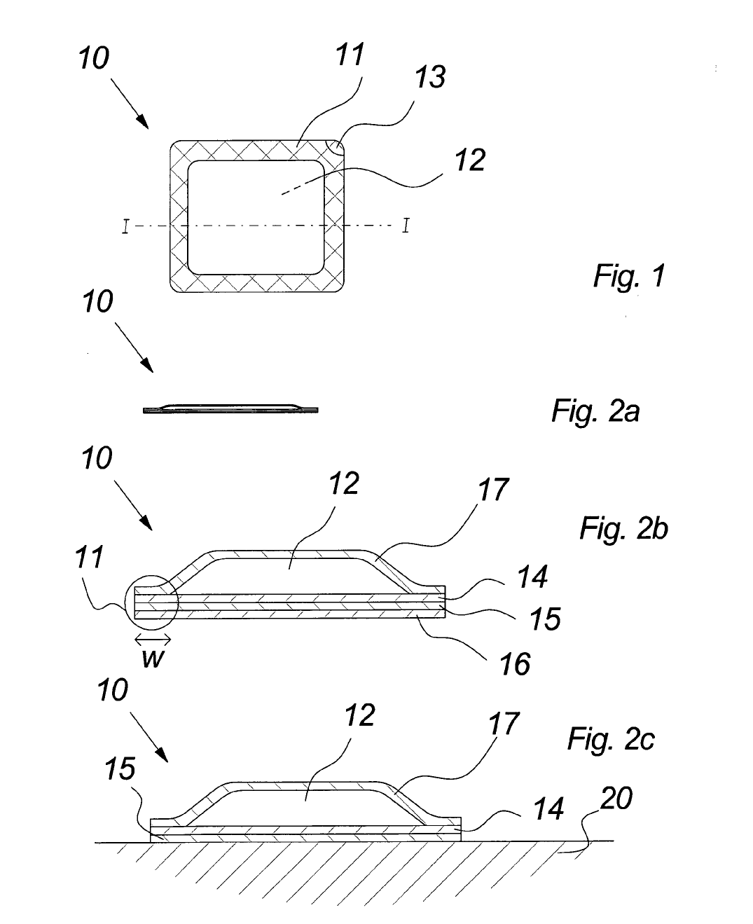

A top view of a transdermal reservoir patch 10 according to an embodiment of the invention is shown in FIG. 1. The backing layer covers the entire upper side of the patch 10, and is thus not shown; see e.g. backing layer 17 on FIG. 2b, which prevents the reservoir content from escaping the reservoir patch through the rear. Moreover, the backing protects the active ingredients of the reservoir against UV radiation and moisture. Moreover, the backing serves as a shield against mechanical impacts on the membrane. The patch 10 comprises a reservoir 12 below the backing layer, a peel strip 13 and a sealing 11. The parts of the patch 10 will be explained further in the following.

The peel strip 13 facilitates removal of the liner 16 from the adhesive 15 prior to mounting on the skin of a user and can be seen in FIG. 2b.

FIG. 2a illustrates a cross sectional view along the line I-I in FIG. 1 of the above-illustrated transdermal reservoir patch 10. In order to distinguish the different layers, a magnification is shown in FIG. 2b from which the layers will be explained. The transdermal reservoir patch 10 comprises an impermeable backing layer 17 which provides an occlusive layer that prevents the content of the reservoir 12 to escape into the environment and to protect the content of the reservoir from being exposed to e.g. humidity or sunlight. The intended path for the content of the reservoir 12 is through the membrane layer 14 and further through the adhesive layer 15 to finally reach through the skin of the user.

The different layers are sealed together giving a sealing 11 with a seal width w.

The impermeable backing layer 17 defines the nonskin facing, or skin distal, side of the patch in use. The material chosen should therefore be nicotine resistant, and should exhibit minimal nicotine permeability. The backing layer should be opaque, because nicotine degrades when exposed to ultraviolet light.

A preferred material is combinations of pigmented polyolefin, aluminized polyester and heat-sealable polyolefinic layers. Polyester has a nicotine permeability less than 0.2 μg.100 μm/cm2.h. Preferred backings are multilayer polyester films, available for example from 3M Corporation as Scotchpak™ 9730.

As relatively few materials are actually really sufficiently impermeable to nicotine to retain the nicotine load adequately during storage or use, other low permeability materials that might be tried include, for example, metal foil, metallized polyfoils, composite foils or films containing polyester, Teflon (polytetrafluoroethylene) type materials, or equivalents thereof that could perform the same function.

The reservoir layer 12 may take various forms, for example, pure nicotine, nicotine diluted with a liquid or immobilized by a gel. The gel can be made from different materials preferably methyl cellulose. The reservoir layer 12 is to be a depot for the nicotine and to keep it in good contact with the membrane layer 14. The reservoir layer 12 does not contribute to any measurable extent to the rate-controlling mechanism.

The content of the reservoir may be any tobacco alkaloid, preferably nicotine.

The term tobacco alkaloid as used herein and in the claims, is taken to mean nicotine or nicotine-like alkaloid such as nor-nicotine, lobeline, and the like, e.g. in the free base or pharmacologically acceptable acid addition salt form. Plant alkaloids of this type are e.g. obtainable from species of Nicotiana which is a source for nicotine and nor-nicotine, as well as species of Lobelia and Lobeliaceae (Indian tobacco) which are a source for lobeline.

The tobacco alkaloid may furthermore be combined with further physiologically active substances which either compensates the physically induced effect of the tobacco alkaloid of the patch or simply adds a further functionality to the patch.

The term “physiologically active substance” as used to describe the principal active ingredient of the device intends a biologically active compound or mixture of compounds that has a therapeutic, prophylactic or other beneficial pharmacological and/or physiological effect on the wearer of the device. Examples of types of drugs that may be used in the inventive device are anti-inflammatory drugs, analgesics, antiarthritic drugs, antispasmodics, antidepressants, antipsychotic drugs, tranquilizers, antianxiety drugs, narcotic antagonists, antiparkinsonism agents, cholinergic agonists, anticancer drugs, immunosuppression agents, antiviral agents, antibiotic agents, appetite suppressants, antiemetics, anticholinergics, antihistamines, antimigraine agents, coronary, cerebral or peripheral vasodilators, hormonal agents, contraceptive agents, antithrombotic agents, diuretics, antihypertensive agents, cardiovascular drugs, nitroglycerine or any other nitrites and or nitrates, scopolamine or combination, oestradiol, progesterone, testosterone, diclofenac, oxibutunin, melatonin, clonodine, lidocaine/lignocaine, ibuprofen, lofexidine, nifedipine, morphine, naloxone, apomorphine, diazepam, 5-flourouracil, buprenorphine, betahistitine, metoclopromide, taxol, cannabis, and the like. The appropriate drugs of such types are capable of permeating through the skin either inherently or by virtue of treatment of the skin with a percutaneous absorption enhancer. Because the size of the device is limited for user-acceptance reasons, the preferred drugs are those that are effective at low concentration in the blood stream. Examples of specific drugs are steroids such as estradiol, progesterone, norgestrel, levonorgestrel, norethindrone, medroxyprogesterone acetate, 3-ketodesogestrel, testosterone and their esters, nitro-compounds such as nitroglycerine and isosorbide nitrates, nicotine, chlorpheniramine, terfenadine, triprolidine, hydrocortisone, oxicam derivatives such as piroxicam, ketoprofen, mucopolysaccharidases such as thiomucase, buprenorphine, fentanyl, naloxone, codeine, dihydroergotamine, pizotiline, salbutamol, terbutaline, prostaglandins such as misoprostol and enprostil, omeprazole, imipramine, benzamides such as metoclopamine, scopolamine, peptides such as growth releasing factor and somatostatin, clonidine, dihydropyridines such as nifedipine, verapamil, ephedrine, pindolol, metoprolol, spironolactone, nicardipine hydrochloride, calcitriol, thiazides such as hydrochlorothiazide, flunarizine, sydononimines such as molsidomine, sulfated polysaccharides such as heparin fractions and the salts of such compounds with pharmaceutically acceptable acids or bases. It should be noted that reservoir patches according to embodiments of the present invention are indeed suitable for delivering one or more active substances chosen from the list above alone or in combination with a tobacco alkaloid.

The membrane layer 14 forms part of the rate-controlling means that regulates the flux of nicotine from the patch to the skin. A suitable material is chosen by considering resistance to attack by nicotine and possession of an appropriate permeability for nicotine. The polymer chosen for the membrane layer 14 should also be compatible with the other components, and workable by standard techniques that are used in fabrication of the patch, such as casting or heat sealing. Dense non-porous membranes have a substantial advantage over micro-porous materials. Micro-porous membranes release the content of the patch by pore flow. Thus, in areas of the pores, the skin is exposed to raw nicotine.

Also, in the case of a volatile liquid such as nicotine, flow through the pores occurs rapidly so that the system is quickly exhausted and the skin is flooded with excess nicotine for the life of the patch. In contrast, diffusion of nicotine through a non-porous film takes place by dissolution of the nicotine in the film, followed by diffusion under a concentration gradient. By selecting materials with suitable permeabilities, and making a membrane of appropriate thickness, it is possible to tailor systems that can release their nicotine load gradually over 12 or 24 hours in a safe, controlled fashion. Furthermore, the solution/diffusion mechanism protects the user's skin from exposure to excess amounts of raw nicotine.

Preferred membrane polymers are low-, medium-, or high-density commercial polyethylenes. Particularly suitable is the membrane obtainable under the trade name CoTran™ 9728 EVA from 3M but other polyethylene membranes faced with adhesive tapes from the 3M Corporation might be very suitable. Other possible membrane materials are polyamides, such as nylon 6,6, or some grades of ethylene vinyl acetate copolymers. Functional equivalents of these are intended to be within the scope of the invention. The membrane layer may be formed by preparing a solution of the chosen polymer in an organic solvent, casting on a glass plate or in a mold, and drying to evaporate the solvent. The thickness of the finished film is tailored to give the desired nicotine flux. A typical thickness of membranes used in transdermal patches range from about 5 μm to about 200 μm. Alternatively, it may be possible to purchase the membrane already in film form. This type of transdermal patch may be prepared by heat-sealing the backing to the membrane layer around the perimeter of the patch.

The nicotine formulation may be added either before or after heat sealing. If the formulation is added before heat sealing, it is convenient to shape the backing so as to form a cavity for retention of the nicotine, or to gel the nicotine. If the formulation is incorporated after heat sealing, the nicotine may be injected into the pouch formed by the heat-sealing process, and the injection hole sealed.

The adhesive layer 15 should be nicotine compatible and permit a useful nicotine flux. In addition, the adhesive should satisfy the general criteria for adhesives used for transdermal patches in terms of biocompatibility, ease of application and removal, etc. Suitable adhesives for use in the practice of the invention include pressure-sensitive adhesives approved for medical use. Amine-resistant types are preferred, so that the adhesive will not be attacked by the nicotine. A range of useful adhesives are offered by Advanced Medical Solutions Ltd. Particularly suitable is the AMS Pressure Sensitive Adhesive No. 10001875. Alternatively, acrylate-type adhesives with amine resistance can be used. The adhesive layer can be cast directly onto the skin-facing side of the membrane or monolith as a thin film. Alternatively, medical adhesive tape, with or without nicotine-flux controlling properties, may be used.

The release liner 16 may be composed of a single layer or a multiplicity of layers. Suitable release liners may be made from materials such as polyester, low-density polyethylene (LDPE), high-density polyethylene (HDPE), polypropylene, polystyrene, polyamide, nylon, polyvinyl chloride and specialty papers, and include Akrosil Biorelease liners, Scotchpak 1022 release liners, Adhesives Research AR5MS, Custom Coating and Laminating 7000 on HDPE or 6020 on polyethylene terephthalase (PET). A treatment of the release liner by e.g. a layer of silicon can advantageously be carried out to prevent an undesired sticking between the adhesive layer 15 and the release liner 16. Generally, the liner should be attached to the membrane with an adhesive force which is less than the adhesive force keeping the membrane to the backing in order to avoid damaging the sealing or the membrane when releasing the liner.

The sealing 11 can be performed by gluing the layers together or preferably by heat sealing the layers. In any case the sealing will have a certain sealing width w as indicated in FIG. 2b. Said sealing width w will have a crucial influence on the functionality of the reservoir patch. In case of a too small sealing width there will be a risk of leakage of the reservoir content sideways through the sealing and into the surroundings. On the other hand, a too big sealing width will cause the reservoir patch to be less comfortable to wear for the user due to the higher stiffness all around the reservoir patch. A higher stiffness like this will also increase the risk of release of a corner or a side of the patch from the skin of the user when placed on a curved surface as for instance an arm or a leg. If a corner or a side of the reservoir patch is released from the skin there will immediately be a risk of the reservoir content to escape to the surroundings. This risk will be higher over time as a released part of a patch will have a tendency to spread and cause an even bigger part of the patch to be released.

FIG. 2c illustrates the patch 10 after release of the liner 16 and placement on a surface 20. This surface may be any surface but is most likely to be the skin of a user of the reservoir patch. The patch is to be placed on the skin, preferably on a skin spot with a small amount of hair and preferably on a place with thin skin. The patch 10 is attached to the skin with the help of the adhesive layer 15.

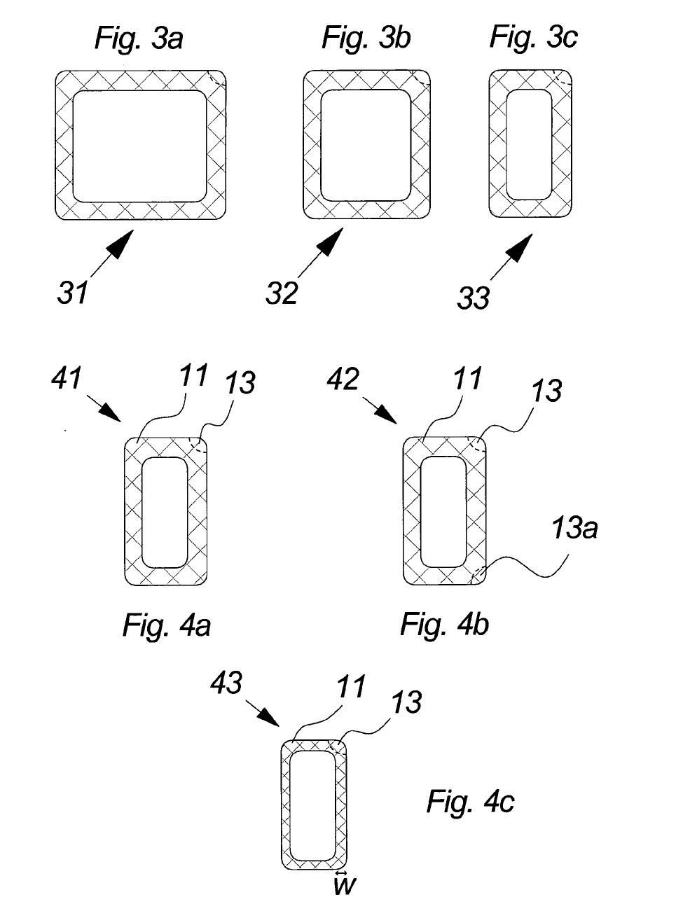

FIGS. 3a, 3b, 3c, 4a, 4b, 5a, 5b, 5c, 5d and 5e illustrate further embodiments according to embodiments of the present invention. FIGS. 3a, 3b and 3c illustrate different sizes 31, 32 and 33 of the reservoir patch as a whole. FIG. 4a illustrates an embodiment of the invention 41. The illustrated patch 41 comprises a sealing 11 which serves to establish a reservoir between a backing and a membrane (not shown). The illustrated patch comprises a peel corner 13. The further patch components may e.g. correspond to the patch components already described in FIGS. 1 to 2c. For reasons of explanation, the patch 41 is referred to as a starting point when explaining further embodiments. It should be noted that numerous variations in size, shape and so on may be applied within the scope of the invention.
FIG. 4b illustrates a patch 42 provided with an extra peel corner 13a. The further patch components may e.g. correspond to the patch components already described in FIGS. 1 to 2c. This variant facilitates easier release of the liner. Further numbers of peel corners, sizes of peel corners or peel areas may be applied within the scope of the invention. Generally, according to embodiments of the invention, peel corners are preferred but optional. FIG. 4c illustrates a further embodiment of the invention 43 where a seal width w of the sealing 11 is decreased in order to increase the flexibility and/or decrease the flexural strength of the patch 43 structure.

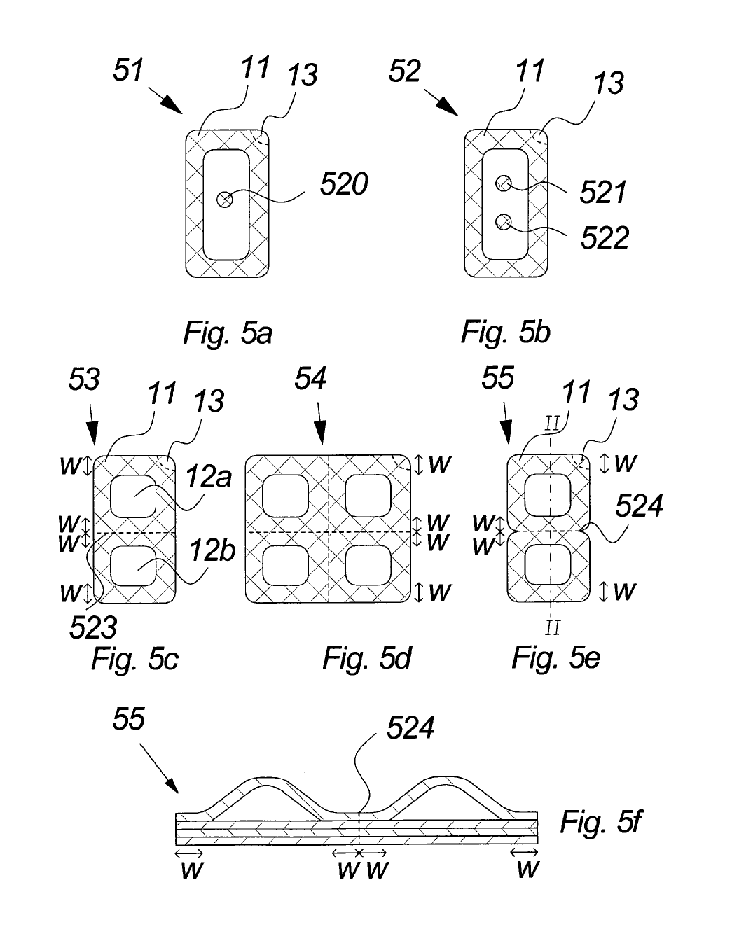

FIGS. 5a-5f illustrate further embodiments according to embodiments of the invention concerning variations in the seal structure. FIG. 5a shows a patch 51 with a sealing 11 and a peel corner 13. The further patch components of the patches in FIGS. 5a to 5f may e.g. correspond to the patch components already described in FIGS. 1 to 2c. The patch 51 is provided with an extra seal spot 520 within the middle of the reservoir in order to stabilize the backing and the reservoir. Stabilization here will keep the shape of the reservoir and maintain the distribution of gel. A further variant is illustrated for the patch 52 in FIG. 5b where two seal spots 521 and 522 are provided. Any number of spots can in this way be provided in order to stabilize the patch according to embodiments of the present invention.

FIG. 5c shows a patch 53 provided with an extra sealing between the membrane and the backing crossing the patch, thereby splitting the patch into two separate patches with separate reservoirs 12a and 12b. The extra sealing is split by a peel line 523 with which the two patches can easily by separated. In this way the patch can be used as normal if just placed on the skin as whole. In addition to that it is possible to split the patch into two separate patches, each of which giving e.g. half the dose, by tearing along the peel line 523. A further embodiment is shown in FIG. 5d illustrating a patch 54 provided with two extra sealings separating the patch into four separate patches each with e.g. one fourth of the total dose. Generally for the patches illustrated in FIGS. 5a to 5e, it should be noted that numerous variations in size, shape and so on may be applied within the scope of the invention.

FIG. 5e shows a further embodiment of the embodiments shown in FIGS. 5c and 5d. A patch 55 where all corners, subject to be exposed when separating the patches, are rounded beforehand in order for the user to avoid sharp corners when separating the patches through the peel line 524. This rounding may preferably be established during manufacturing of the patch.

FIG. 5f shows a cross-sectional view along the line II-II of the patch 55 in FIG. 5e and might as well have been of any the patches in FIGS. 5c and 5d. The widths of the reservoir defining seals are marked w.

Different physiologically active substances show very varying ability to penetrate the skin. Nicotine easily permeates the skin, whereas other physiologically active substances, typically larger in molecule size, must be assisted in order to get a noticeable amount of ingredient through the skin. For this purpose enhancers are used in an embodiment of the invention.

Suitable penetration enhancers, flux enhancers, are preferably monovalent, saturated or unsaturated aliphatic, cycloaliphatic or aromatic alcohols having from 4 to 12 carbon atoms. e.g. n-hexanol or cyclohexanol, aliphatic, cycloaliphatic or aromatic hydrocarbons having from 5 to 12 carbon atoms, e.g. hexane, cyclohexane, isopropylbenzene and the like, cyclo-aliphatic or aromatic aldehydes and ketones having from 4 to 10 carbon atoms, such as cyclohexanone, acetamide, N,N-di-lower alkylacetamides such as N,N-dimethylacetamide or N,N-diethyl-acetamide, c.sub.10-c.sub.20-alkanoylamides, e.g. N,N-dimethyllauroylamide, 1-n-C.sub.10-c.sub.20-alkylazcycloheptan-2-one, e.g. 1-n-dodeclyazacycloheptan-2-one(Azone.RTM. laurocapram), or N-2-hydroxyethylacetamide, and known vehicles and/or penetration enhancers such as aliphatic, cycloaliphatic and aromatic esters N,N-di-lower alkylsulphoxides, unsaturated oils, halogenated or nitrated aliphatic or cyclo-aliphatic hydrocarbons, salicylates, polyalkylene glycol silicates, and mixtures thereof.

The following examples will further illustrate embodiments of the present invention. It is to be understood that the examples set forth are illustrative and not limiting for the present invention.

### EXAMPLE 1

A reservoir patch, with backing layer, membrane and adhesive according to the description, with patch delivering area of 3.2 cm² was manufactured comprising 20 mg of nicotine mixed in 170 mg of gelling agent in the reservoir. In this sense the resulting patch had a ratio between the tobacco alkaloid content and the patch delivering area of 6.25 mg/cm².

The patch with this ratio between nicotine present in the reservoir and patch delivering area proved to be acceptable.

### EXAMPLE 2

A series of reservoir patches with identical patch delivering area of 9.52 cm² was manufactured, the data of which are shown in table 1.

TABLE 1

| | Gel content | Nicotine | | Area | Flux |
|---|---|---|---|---|---|
| Patch | (mg) | content (mg) | Type | (cm²) | (mg/h * cm²) |
| A | 190 | 19 | 15 mg/16 h | 9.52 | 0.10 |
| B | 260 | 26 | 21 mg/24 h | 9.52 | 0.09 |
| C | 190 | 26.6 | 21 mg/24 h | 9.52 | 0.09 |
| D | 285 | 39.9 | 31.5 mg/24 h | 9.52 | 0.14 |
| E | 500 | 50 | 40 mg/24 h | 9.52 | 0.18 |
| F | 1000 | 100 | 80 mg/24 h | 9.52 | 0.35 |
| G | 1500 | 150 | 120 mg/24 h | 9.52 | 0.53 |
| H | 1500 | 210 | 165 mg/24 h | 9.52 | 0.72 |
| I | 1500 | 300 | 237 mg/24 h | 9.52 | 1.04 |

All patches A-I proved to be acceptable for containing a deliverance of nicotine and for an effective fastening on the human body. In table 1 flux is calculated as delivered nicotine per time per patch delivering area instead of total nicotine content per time per patch delivering area. Thereby this indicates the actual amount delivered, i.e. for patch A the flux is 15 mg/16 h*cm²=0.10 mg/h*cm².

### EXAMPLE 3

A series of reservoir patches as in example 1 with higher ratios were manufactured. The series consisted of ratios of app. 10, 20, 40, 60, 80, 100 mg/cm². The patches proved to be acceptable for ratios as high as 100 mg/cm².

### EXAMPLE 4

A series of reservoir patches as in example 1 with increased patch area were manufactured with patch delivering areas of 3.2 cm², 6.3 cm², 9.52 cm², 14.3 cm², 20.1 cm², 27.6 cm² and 35.7 cm². A larger size of patch made it possible to contain and deliver a larger amount of nicotine and reducing the risk that the nicotine caused burning of the skin of the user.

### EXAMPLE 5

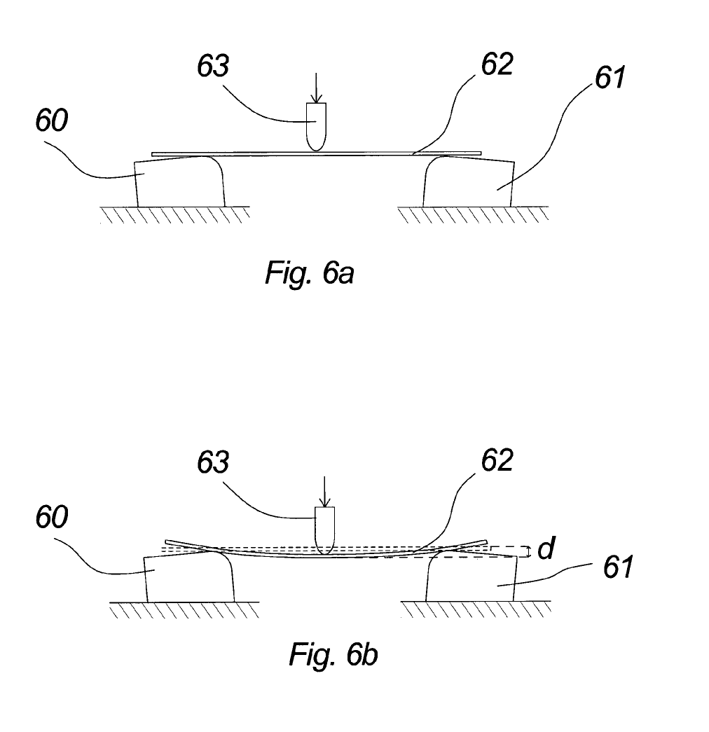

The tests for flexural strength referred to in the description are based on the ISO 178 standard unless otherwise stated. The test measures the force required to bend a material under 3 point loading conditions as illustrated in FIGS. 6a and 6b. A material 62 for which the flexural strength is to be measured is placed on a support span provided by support structures 60 and 61. In the middle of the material 62 a roll 63 presses down on the material and the necessary force to move the material d mm down is measured within a certain range.
The separation between the support structures 60 and 61 was 30 mm. The velocity of the roll 63 to bend the material 62 was 5 mm/min. The values were found by measuring the necessary force to move the material downwards from d=1 mm to d=5 mm. References to flexural strength of patches according to embodiments of the

[symbol]

1. A tobacco alkaloid patch (10; 41; 42; 43; 51; 52; 53; 54) for transdermal administration of a tobacco alkaloid, said patch comprising an impermeable backing (11) and a membrane (14) which defines a reservoir (12) there between, said membrane being permeable to and in contact with said tobacco alkaloid, said tobacco alkaloid being mixed with a viscous flowable gel, said impermeable backing and said membrane being at least partly joined by a sealing and wherein the amount of said tobacco alkaloid is above 10 mg.

2. A tobacco alkaloid patch according to claim 1, wherein the amount of said tobacco alkaloid is above 20 mg, 40 mg, or 60 mg, is preferably above 80 mg, and most preferably above 100 mg.

3. A tobacco alkaloid patch according to claim 1, wherein the initial flux of said tobacco alkaloid through the membrane is above 10 μg/cm² h, above 50 μg/cm² h, above 100 μg/cm² h, or is above 400 μg/cm² h.

4. A tobacco alkaloid patch according to claim 1, wherein
the ratio between said tobacco alkaloid in the patch and the patch delivering area is above 1 mg/cm² or is above 2.5 mg/cm²; and/or
the ratio between the delivered tobacco alkaloid and the patch delivering area is above 0.5 mg/cm², above 1 mg/cm², above 1.5 mg/cm², or above 2.5 mg/cm².

5. A tobacco alkaloid patch according to claim 1, wherein
the nicotine permeability of said membrane is above 20 μg·100 μm/cm²·hr, above 50 μg·100 μm/cm²·hr or above 220 μg·100 μm/cm²·hr; and/or
said sealing comprises a glue sealing or a heat sealing.

6. A tobacco alkaloid patch according to claim 1, wherein
the width of said sealing is at least 1 mm, preferably at least 1.5 mm, at least 2.0 mm, or is at most 10.0 mm; and/or
said membrane and said backing are joined at the circumference of the patch; and/or
wherein said sealing comprises circumferential sealing plus one or more sealing dots therein between plus, optionally, a pattern of any kind.

7. A tobacco alkaloid patch according to claim 1, wherein said patch comprises a detachable release liner (16), which optionally: comprises one or more peelable corners and/or is attached to the membrane of the patch by an adhesive force which is weaker than the adhesive force obtained by said sealing between said membrane and said backing.

8. A tobacco alkaloid patch according to claim 1, wherein said tobacco alkaloid comprises nicotine.

9. A tobacco alkaloid patch according to claim 1, wherein said patch delivers more than about 50%, 60% 70% of the total content of tobacco alkaloid to the user within 24 hours from releasing the liner and placing the patch on the skin of the user.

10. A tobacco alkaloid patch according to claim 1, wherein
said impermeable backing further comprises permeable layers, said layers may be placed on the inner side and/or on the outer side of the impermeable backing; and/or
said impermeable backing is made of a multilayer film, such as a multilayer polyester film, or is made of combinations of one or more of the following; pigmented polyolefin, aluminized polyester, metal foil and heat-sealable polyolefinic layers; and/or
said impermeable backing has a thickness between 1 μm and 500 μm, between 5 μm and 200 μm, or between 10 μm and 100 μm.

11. A tobacco alkaloid patch according to claim 1, wherein
said membrane comprises a polyethylene membrane, which optionally comprises polyamides, such as nylon 6,6, or some grades of ethylene vinyl acetate copolymers or functional equivalents of these; and/or
said membrane is non-porous.

12. A tobacco alkaloid patch according to claim 1, wherein
said patch comprises an adhesive, which is optionally substantially equally distributed all over the lower part of the patch and optionally further comprises an adhesive pattern; and/or
said adhesive has a thickness of below 3 mm, below 1 mm, above 0.01 μm, or above 0.1 μm; and/or
an adhesive layer is attached to said membrane in order to attach the patch to the skin of a user.

13. A tobacco alkaloid patch according to claim 1, wherein
said cavity forms a reservoir for said tobacco alkaloid; and/or
said tobacco alkaloid is contained within said reservoir in liquid form; and/or
said tobacco alkaloid is confined between said impermeable backing and said membrane within said reservoir substantially immobilized by a viscous flowable gel.

14. A tobacco alkaloid patch according to claim 1, wherein
said reservoir contains tobacco alkaloid in an amount of more than 0.5 mg, and optionally, a gelling agent in an amount of less than 20 mg or less than 10 mg; optionally
purified water in an amount of less than 1000 mg or less than 800 mg; or wherein said reservoir optionally contains tobacco alkaloid, gelling agent and purified water in a total amount of less than 1000 mg or less than 800 mg; and/or
said reservoir is shaped in a rectangular, circular or oval form, and/or is shaped in the form of a donut or in an essentially rectangular form with rounded corners; and/or
said reservoir is made up of several minor reservoirs separated by sealing; and/or
said reservoir has a volume of less than 4000 mm³, preferably less than 3000 mm³, less than 2000 mm³, preferably less than 1500 mm³, or more than 50 mm³, preferably more than 75 mm³.

15. A tobacco alkaloid patch according to claim 1, wherein
said patch can be separated into two or more separate reservoir patches, border region(s) between said two or more reservoir patches optionally comprising predefined peel-lines (523) and, optionally, corners of said two or more separate reservoir patches are pre-rounded, the patch being optionally separated into two or more separate patches with rounded corners.

16. A tobacco alkaloid patch according to claim 1, wherein said reservoir comprises a flux-controlling gel, optionally constituted by purified water and a gelling agent selected from the group of hyroxyethyl starch, dextran, hydroxyethylcellulose (HEC), hydroxypropylmethylcellulose (HPMC), hydroxypropycellulose (HPC), methylcellulose (MC), hydroxyethyl-methylcellulose (HEMC), ethylhydroxyethylcellulose (EHEC), carboxymethyl cellulose (CMC), poly(vinyl alcohol), poly(ethylene oxide), poly(2-hydroxyethylmethacrylate), pyrolidone), and pluronics.

17. A tobacco alkaloid patch according to claim 16, wherein said gelling agent is hydroxypropyl methyl cellulose or methyl cellulose.

18. A tobacco alkaloid patch according to claim 1, wherein
said patch is coloured in order to match the skin of a user or is essentially transparent and is optionally produced with a pattern or a picture on the backing; and/or
the total size of said patch is less than 40 cm² or less than 25 cm², or is above 1 cm² or above 2 cm².

19. A tobacco alkaloid patch according to claim 1, wherein
said reservoir further contains another active ingredient and optionally one or more enhancers added to enhance the uptake of said other active ingredient, said enhancers being optionally present in an amount of less than 2000 mg; and/or
said patch has a flexural strength of less than about 250 mN/mm or less than about 50 mN/mm.

20. A tobacco alkaloid patch according to claim 1, wherein
said impermeable backing (17) has a flexural strength which is greater than the flexural strength of the membrane (14); and/or
said impermeable backing has a flexural strength of at least 0.5 mN/mm or at least 1 mN/mm; optionally the flexural strength of the impermeable backing is at least 0.5 mN/mm and wherein said patch has a flexural strength of less than about 50 mN/mm; or optionally the flexural strength of the impermeable backing is at least 0.5 mN/mm and wherein said patch has a flexural strength of less than about 40 mN/mm; or optionally the impermeable backing of said patch has a flexural strength of at least less than about 30 mN/mm; optionally said patch has a flexural strength of less than about 25 mN/mm; or optionally said patch has a flexural strength of about 1 to 30 mN/mm.

21. A tobacco alkaloid patch according to claim 1, wherein
the backing (17) comprises a multilayer structure; and/or.
said tobacco alkaloid patch is a nicotine reservoir patch; and/or
wherein said patch has a flexural strength greater than of about 2 mN/mm; and/or.
said tobacco alkaloid is above 110 mg, preferably above 160 mg, most preferably above 210 mg; and/or
said patch has a (reservoir volume)/(delivering area) ratio of more than 1 mm³/cm².

22. A tobacco alkaloid patch according to claim 1, packed in a controlled atmosphere, which is optionally nitrogen.
